(19) United States
(12) Patent Application Publication
(10) Pub. No.: US 2009/0105742 A1
(43) Pub. Date: Apr. 23, 2009

(54) TRANSSEPTAL GUIDEWIRE

(75) Inventors: Paul Kurth, Santa Barbara, CA (US); Andrew W. Armour, Swarthmore, PA (US)

Correspondence Address: RATNERPRESTIA, P.O. BOX 980, VALLEY FORGE, PA, 19482, US

(73) Assignee: PRESSURE PRODUCTS MEDICAL SUPPLIES INC., San Pedro, CA (US)

(21) Appl. No.: 11/875,365

(22) Filed: Oct. 19, 2007

(51) Int. Cl. A61M 25/09; A61B 17/34

(52) U.S. Cl. 606/185; 604/164.13

(57) ABSTRACT

A transseptal guidewire and methods for perforating the intra-atrial septum of the heart are disclosed. The transseptal guidewire has an elongated body with an end section and a tapered distal section. At least a portion of the end section has a first dimension in a first direction transverse to a longitudinal axis of the elongated body. The first dimension is larger than a second dimension of the portion of the end section in a second direction transverse to the longitudinal axis.

### FIELD OF THE INVENTION

The present invention relates generally to less invasive surgical equipment and surgical procedures. More particularly, the present invention relates to devices and methods for crossing from the right atrium to the left atrium by perforating the intra-atrial septum of the heart for the treatment of intracardiac arrhythmias and defects such as, for example, atrial fibrillation and valve defects related to cardiac disease as well as for pacing, ablating, and correction of other structural defects.

### BACKGROUND OF THE INVENTION

Since the 1950's, transseptal procedures of the heart have been traditionally performed using Brockenbrough needles in which a puncture is made through an intact atrial septum from the right atrium to the left atrium. Several risks, however, have been associated with the use of Brockenbrough needles. One risk is the perforation of the lateral atrial wall after crossing the atrial septum. Another risk is the potential perforation of the aortic root.

Attempts have been made to reduce these and other risks. For example, U.S. Pat. No. 5,312,341 relates to the problem of inadvertent withdrawal of a catheter tip from the left atrium, through the atrial septum, and back into the right atrium. A retaining means for retaining the distal tip of a sheath which has been placed through a septum, such as the interatrial septum, across the septum, in the left atrium during left heart procedures was therefore proposed.

U.S. Pat. No. 6,650,923 relates to a method for accessing the left atrium by locating the fossa ovalis of the intra-atrial septum. An access catheter with a detector for identifying and providing access through the fossa ovalis was proposed.

U.S. Patent Publication No. 2006/0064062 relates to transseptal puncture needles and transseptal puncture needle assemblies. More specifically, it relates to curved transseptal puncture needles and needle assemblies that facilitate insertion through curved transseptal introducers. Each curved transseptal puncture needle includes a needle tip with a tangential back bevel configuration, a reverse tangential back bevel configuration, or a conical reverse bevel configuration.

U.S. Patent Publication No. 2005/0101984 relates to septal puncture in patients in which a communication is present between the two atria of the heart, for example, a patient with a patent foramen ovale (PFO). A device and method are proposed to safely puncture both an intact atrial septum and an atrial septum having a PFO. The proposed device includes a blunt outer needle, and a second inner needle disposed longitudinally through the lumen of the outer needle, wherein the inner needle is flexible, e.g., has a flexible portion and/or a bend or other non-traumatic conformation at its tip.

U.S. Patent Publication Nos. 2005/0159738 and 2005/0065507 relate to devices for septal perforation utilizing radio frequency energy. Each device includes a functional tip with at least one active electrode capable of creating a controlled perforation in body tissue. The device is introduced into the right atrium and the functional tip is positioned against the atrial septum. Energy is applied to the tip to create the perforation.

U.S. Pat. No. 6,890,353 relates to a method and apparatus for reducing mitral regurgitation by applying a force to the wall of the coronary sinus so as to force the posterior leaflet anteriorly and thereby reduce mitral regurgitation. A guidewire uses a sharp tip for allowing the distal end of a guidewire to penetrate tissue.

U.S. Patent Publication No. 2006/0241648 relates to methods and apparatus for modifying tissue. The proposed method includes advancing a beveled distal tip of a guide member to facilitate advancement of the guide member through tissue. A modification device is advanced along the guide member.

Nevertheless, there remains a need for improved devices and methods for perforating the intra-atrial septum of the heart with devices that improve the safety of the procedure.

### SUMMARY OF THE INVENTION

In one aspect, the invention provides a transseptal guidewire configured to perforate the intra-atrial septum. The transseptal guidewire has an elongated body with an end section and a tapered distal section. At least a portion of the end section has a first dimension in a first direction transverse to a longitudinal axis of the elongated body. The first dimension is larger than a second dimension of the portion of the end section in a second direction transverse to the longitudinal axis.

In another aspect, the invention provides a transseptal guidewire having an elongated body with an end section biased to a curved configuration and a tapered distal section. The elongate body has an imagable section proximal of the end section, and the imagable section includes at least one radiopaque marker. At least a portion of the end section has a first dimension in a first direction transverse to a longitudinal axis of the elongated body that is larger than a second dimension in a second direction transverse to the longitudinal axis.

In yet another aspect, a method of fabricating a transseptal guidewire is provided. The method includes coupling at least one radiopaque marker to an elongate body to form an imagable section of the elongate body, ovalizing at least a portion of an end section of the elongate body distal of the imagable section, and heat curving at least a portion of the end section to a provide a curved configuration.

In still yet another aspect, a method of confirming traversal of an intra-atrial septum is provided. The method includes perforating the intra-atrial septum from a right atrium of a heart to a left atrium of the heart using a transseptal guidewire, extending at least a portion of an end section of the transseptal guidewire into the left atrium, and imaging an imagable section of the transseptal guidewire proximal of the end section in the left atrium by means of at least one radiopaque marker coupled to the transseptal guidewire at the imagable section.

In another aspect, a method of providing vascular access to the left atrium is provided. The method includes perforating the intra-atrial septum from a right atrium of a heart to a left atrium of the heart using a transseptal guidewire, extending at least a portion of an end section of the transseptal guidewire into the left atrium, and imaging an imagable section of the transseptal guidewire proximal of the end section in the left atrium by means of at least one radiopaque marker coupled to the transseptal guidewire at the imagable section. The method further includes advancing the transseptal guidewire into one of the pulmonary veins to confirm location and advancing a transseptal introducer over the transseptal guidewire into the left atrium.

### BRIEF DESCRIPTION OF THE DRAWING

The invention is best understood from the following detailed description when read in connection with the accompanying drawings, with like elements having the same reference numerals. This emphasizes that according to common practice, the various features of the drawings are not drawn to scale. On the contrary, the dimensions of the various features are arbitrarily expanded or reduced for clarity. Included in the drawings are the following figures:

FIG. 1 is a schematic representation of a heart showing an embodiment of a transseptal trocar device positioned within the heart;

FIG. 2 is a cross-sectional side view of the transseptal trocar device shown in FIG. 1;

FIG. 3 is a perspective view of a transseptal guidewire of the transseptal trocar device according to one exemplary embodiment of the invention;

FIG. 4A is a side view of the transseptal guidewire of FIG. 3 in an intermediate stage of fabrication according to an exemplary method of fabricating the transseptal guidewire;

FIG. 4B is an enlarged view of a tapered portion of the transseptal guidewire shown in FIG. 4A;

FIG. 4C is an enlarged view of a tapered distal section of the transseptal guidewire shown in FIG. 4A;

FIG. 5 is a side view of the transseptal guidewire illustrated in FIG. 4A in another intermediate stage of fabrication according to an exemplary method of fabricating the transseptal guidewire;

FIG. 6 is a side view of the transseptal guidewire illustrated in FIGS. 4A and 5 according to an exemplary method of fabricating the transseptal guidewire;

FIG. 7A is a cross-sectional view of an embodiment of an imagable section of the transseptal guidewire shown in FIG. 5 along lines 7A-7A;

FIG. 7B is a cross-sectional view of an embodiment of an ovalized portion of an end section of the transseptal guidewire shown in FIG. 5 along lines 7B-7B;

FIG. 8 is a perspective cross-sectional view of an embodiment of the end section of the transseptal guidewire; and

FIG. 9 is a schematic representation of the heart showing vascular access of the transseptal guidewire to the left atrium.

### DETAILED DESCRIPTION OF THE INVENTION

Aspects of the invention will now be described with reference to the figures. Such figures are intended to be illustrative rather than limiting and are included herewith to facilitate the explanation of the present invention.

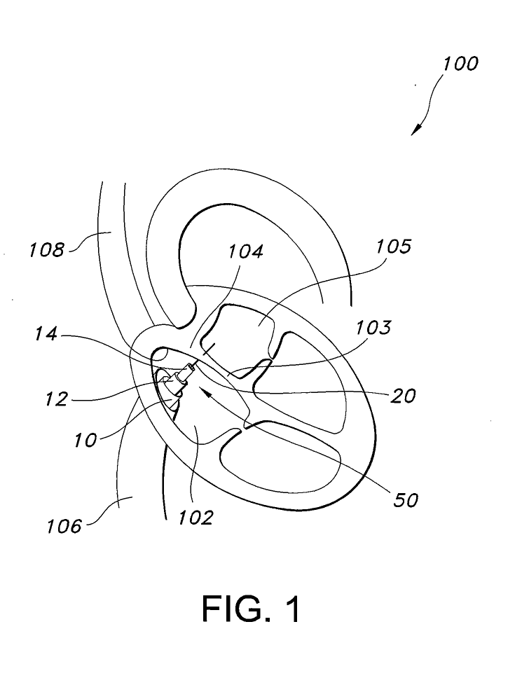

Referring generally to the figures (FIGS. 1-9), in accordance with an exemplary embodiment, a transseptal guidewire 20 configured to perforate the intra-atrial septum 104 of the heart 100 is provided. The transseptal guidewire 20 has an elongated body 22, an end section 26, and a tapered distal section 28. At least a portion of the end section 26 has a first dimension X in a first direction transverse to a longitudinal axis 1 of the elongated body 22 that is larger than a second dimension Y in a second direction transverse to the longitudinal axis 1. In an exemplary embodiment, when the transseptal guidewire 20 perforates the intra-atrial septum 104 and extends into the left atrium 105, the end section 26 is biased in a curved configuration to help render end section 26 atraumatic so as to prevent perforation of the left atrial wall.

Referring now to the individual figures in detail, FIG. 1 depicts a schematic representation of a heart 100 having a transseptal trocar device 50 positioned within heart 100. The transseptal trocar device 50 is configured to perform transseptal catheterizations for access into the left atrium 105 of heart 100 from the right atrium 102 by way of either the inferior vena cava 106 or superior vena cava 108 which supply blood into the right atrium 102 of heart 100.

By a method described in greater detail below, the transseptal trocar device 50, which includes a transseptal sheath 10, dilator 12, outer needle 14, and transseptal guidewire 20, is placed against a septum, such as the intra-atrial septum 104. In an exemplary embodiment, when the distal tip of outer needle 14 is properly positioned in contact with the thin walled fossa ovalis 103 of the intra-atrial septum 104, transseptal guidewire 20 is abruptly extended from the lumen of outer needle 14 to perforate the fossa ovalis 103. Following penetration of the intra-atrial septum 104, and without changing the position of outer needle 14, the distal tip of dilator 12, along with the distal tip of transseptal sheath 10 is passed through the septum and into the left atrium 105.

At times, dilator 12 and sheath 10 do not have sufficient stiffness to pass through the perforation hole (not shown) made in the fossa ovalis 103. In such instances, outer needle 14 may be passed over the guidewire 20 to dilate the septum prior to inserting dilator 12 and sheath 10 through the perforation. The outer needle 14 can also provide support while dilator 12 and sheath 10 are advanced beyond outer needle 14 and through the perforation hole into the left atrium 105. In another embodiment, dilator 12 is optionally made from material that provides sufficient support during the transseptal perforation procedure and the outer needle 14 may not be needed and can be eliminated from device 50.

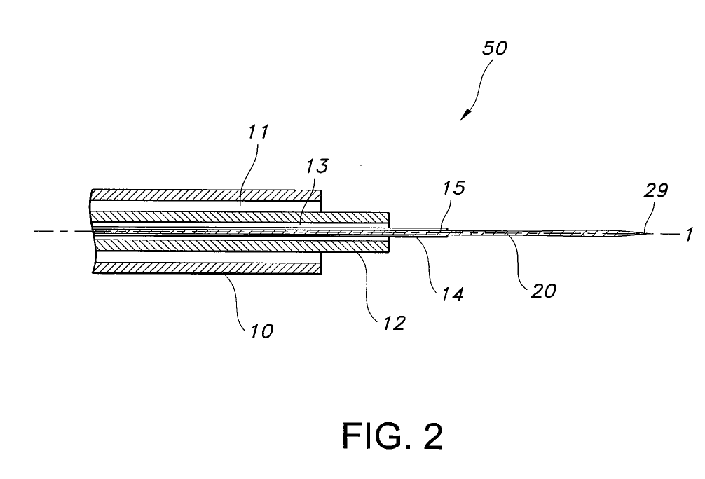

Referring now to FIGS. 1 and 2, aspects of the transseptal trocar device 50 will be described in further detail. Transseptal trocar device 50 includes a transcutaneous intravascular sheath 10 through which components of the device 50 pass from outside the patient's body through a vessel, for example, the femoral vein, through the inferior vena cava 106 into the right atrium 102. Alternatively, sheath 10 may be advanced through a vessel located at an upper half of the body, such as the subclavian vein, through the superior vena cava 108 into the right atrium 102. The sheath 10 and/or other components of transseptal trocar device 50 may have a fixed curve or may be steerable by actuators on a control handle (not shown) located at a proximal end of sheath 10 to aid in delivering the device 50 along the tortuous vascular path leading to the patient's right atrium 102.

According to an exemplary embodiment, sheath 10 is made from soft polymer materials such that sheath 10 is pliable and atraumatic when advanced through vasculature. For example, polymers such as polyimide, polyamide, polyetherblockamide, polyethylene, polytetrafluoroethylene (PTFE), fluorinated ethylene propylene (FEP), and polyurethane may be used. Other biocompatible polymer materials that minimize damage to tissue during the delivery of device 50 to the right atrium 102 may also be used. Transseptal trocar device 50 also includes a dilator 12 slidingly positioned within a sheath lumen 11 of sheath 10 axially disposed along longitudinal axis 1. Dilator 12 is configured to dilate a perforation hole (not shown) made in the intra-atrial septum 104 to provide improved access for the sheath 10 into the left atrium 105. In an exemplary embodiment, the distal end of dilator 12 may be blunted or tapered (not shown) toward outer needle 14 to provide gradual dilation of the perforation hole as dilator 12 is slidingly advanced into the left atrium 105.

As shown in FIG. 2, dilator 12 includes a lumen 13 which receives outer needle 14. Outer needle 14 has a distal end that contacts intra-atrial septum 104 to position outer needle 14 against fossa ovalis 103. In an exemplary embodiment, outer needle 14 is similar in size to a Brockenbrough needle, e.g., with tip diameter of about 0.8 mm. Outer-needle 104 also includes a lumen 15 to receive and provide structural columnar support for a septal perforator, such as a transseptal guidewire 20 axially disposed within lumen 15. The inner diameter of lumen 15 typically approximates the maximum outer diameter of transseptal guidewire 20 such that the transseptal guidewire 20 is slidable within outer needle 14. In certain embodiments, the outer diameter of outer needle 14 gradually tapers toward transseptal guidewire 20 to also function as a dilator of the perforation hole (not shown) created in the fossa ovalis 103.

According to an exemplary embodiment, dilator 12 and outer needle 14 may be made of a polymer material, as described above. Other materials that provide sufficient support during the transseptal procedure are contemplated. For example, various metals, such as nitinol, steel, or titanium, or alloys thereof may be used.

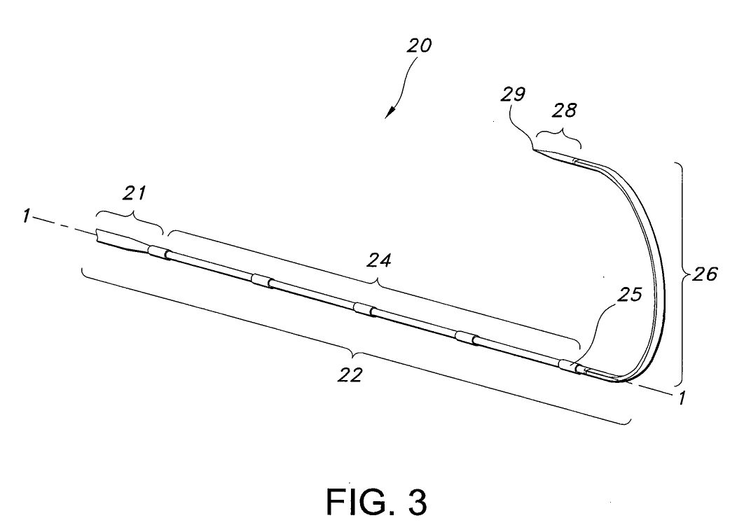

Referring now to FIGS. 2 and 3, aspects of the transseptal guidewire 20 according to one exemplary embodiment of the present invention are described in further detail. Transseptal guidewire 20 is configured to perforate the intra-atrial septum 104 and is disposed within lumen 15 so that transseptal guidewire 20 is reciprocally and axially moveable within outer needle 14. If necessary, the transseptal guidewire 20 can be rotated as well. Transseptal guidewire 20 has a length longer than outer needle 14, for example about 15 cm longer than outer needle 14 such that transseptal guidewire 20 has an overall length that is longer than about 117.5 cm, though other dimensions are contemplated as well. In another embodiment, transseptal guidewire 20 has a length that is about 50 cm longer than outer needle 14. The diameter of transseptal guidewire 20 is sized to fit through the lumen 15 of commercially available transseptal outer needles 14. For instance, the diameter of transseptal guidewire 20 is less than the diameter of lumen 15 of outer needle 14 so transseptal guidewire 20 may pass through outer needle 14 with little or no resistance.

Now referring to FIG. 3, the transseptal guidewire 20 includes an elongated body 22, an end section 26, and a tapered distal section 28. The tapered distal section 28 terminates at a pointed tip 29 at the distal end of transseptal guidewire 20. In an exemplary embodiment, when transseptal guidewire 20 is positioned fully within outer needle 14, transseptal guidewire 20 retains a substantially straight configuration. When transseptal guidewire 20 extends through the distal end of lumen 15 along longitudinal axis 1, end section 26 is no longer supported within outer needle 14 and flexes to a curved conformation such as that shown for illustration purposes in FIG. 3. Thus, in use, when the distal end of outer needle 14 contacts intra-atrial septum 104, transseptal guidewire 20 may extend so that pointed tip 29 perforates intra-atrial septum 104 and is positioned in left atrium 105. As transseptal guidewire 20 continues its path along axis 1 into left atrium 105, end section 26 curves into a non-traumatic conformation so that the lateral wall of the left atrium 105 is not exposed to pointed tip 29. Preferably, the tip 29 is sufficiently flexible so that it does not need the curve to be atraumatic.

In an exemplary embodiment, transseptal guidewire 20 may be coated with a material to ease insertion through the lumen 15 of commercially available transseptal outer needles 14 and/or to prevent clots from forming on the guidewire 20. For example, the entire length of transseptal guidewire 20 or a portion of its length may be coated with a material that has antithrombogenic properties to prevent clots from forming on the wire. Exemplary coatings may be hydrophobic or hydrophilic. Typical coatings may be formed from Teflon, a silicone fluid, or urethane based polymers. Other biocompatible coatings that provide the above mentioned properties may also be used.

As will be described in further detail below, a portion of end section 26 is ovalized such that end section 26 has a substantially non-circular cross section. Ovalizing a portion of end section 26 partly assists with biasing end section 26 in a curved configuration such as that shown in FIG. 3.

As also illustrated in FIG. 3, elongated body 22 of transseptal guidewire 20 has a portion 24 proximal of end section 26. Portion 24 has a substantially circular cross section relative to end section 26. In one embodiment, portion 24 is an imagable section having radiopaque markers 25a-e coupled to the imagable section 24. Radiopaque markers 25a-e may be made of a platinum/iridium alloy and are sufficiently visible under fluoroscopy (x-ray) to assist with imaging of the operative area. In one embodiment, the radiopaque markers 25a-e are formed by a platinum coating or cladding. Other radiopaque materials may also be used such as gold, silver, tungsten, etc.

In an exemplary embodiment, when a portion of imagable section 24 extends into the left atrium 105 from the perforation hole (not shown), x-ray imaging of radiopaque markers 25a-e may confirm successful perforation of the intra-atrial septum 104. Radiopacity of markers 25a-e is generally equal to, or greater than, transseptal needle 20, thus eliminating the need for radiopaque contrast solution.

Radiopaque markers 25a-e are retained on imagable section 24 since end section 26 is ovalized and has a dimension (such as a width) greater than the diameter of imagable section 24. Additionally, a portion 21 of elongated body 22 proximal to imagable section 24 has a tapered transition to imagable section 24 with a diameter also greater than the diameter of imagable section 24. Thus, radiopaque markers 25a-e are retained to imagable section 24 between adjacent portion 21 and end section 26.

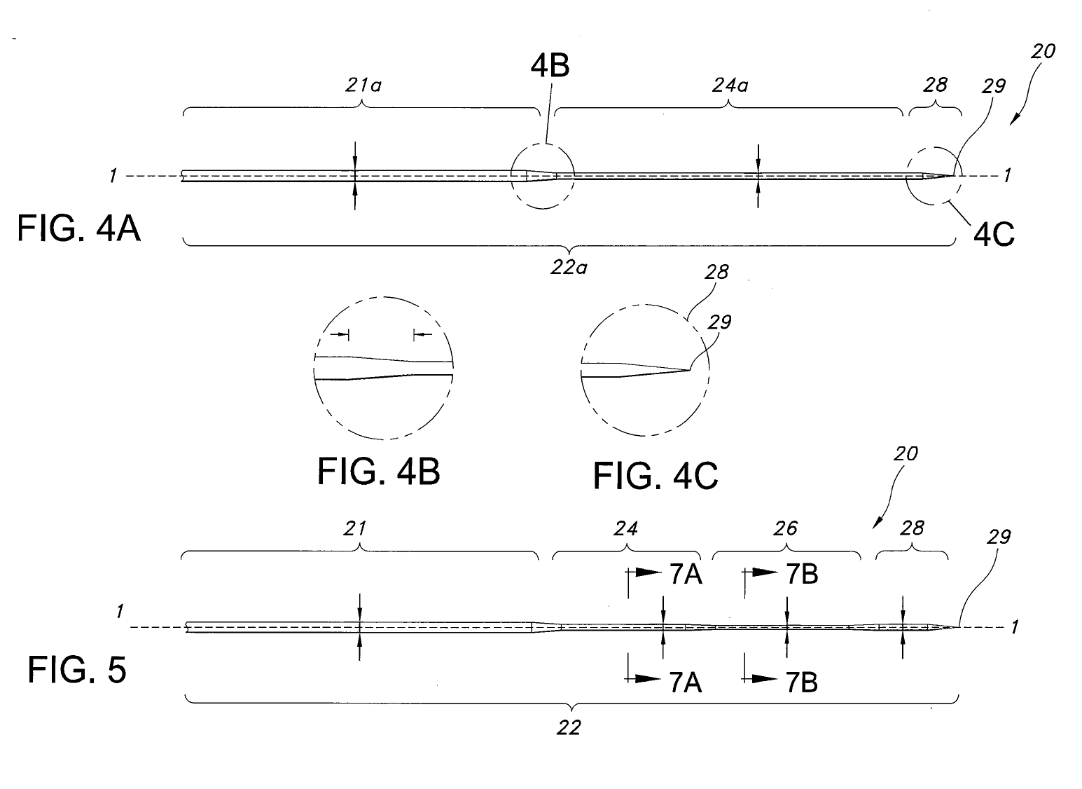

Referring now to FIGS. 4A-C, 5, and 6, a method of fabricating a transseptal guidewire 20 of the present invention is illustrated. As shown in FIG. 4A, which illustrates an intermediate configuration of transseptal guidewire 20 during the manufacturing process, transseptal guidewire 20 has an elongate body 22a disposed along longitudinal axis 1 of transseptal guidewire 20. Transseptal guidewire 20 is optionally made from various metals such as, for example, nitinol, steel, or titanium, or alloys thereof or polymers such as polyimide, polyetheretherketone (PEEK), polyamide, polyetherblockamide, polyethylene, polytetrafluoroethylene (PTFE), fluorinated ethylene propylene (FEP), and polyurethane. In an exemplary embodiment, transseptal guidewire 20 is manufactured from superelastic nitinol wire from Fort Wayne Metals in Fort Wayne, Ind.

In one embodiment, superelastic nitinol wire having a substantially circular cross-section is formed into elongate body 22a by a centerless grinding process. The superelastic nitinol wire, for example, may have a substantially uniform diameter and is threaded into a grinding machine to gradually decrease the diameter of the wire. Elongate body 22a may have a maximum diameter of about 0.015 inches at a proximal portion 21a which is tapered by centerless grinding to portion 24a. Portion 24a is sharpened to tapered distal section 28 terminating at pointed tip 29. Pointed tip 29 has a substantially circular cross-section and is positioned at the distal end of tapered distal section 28.

In another embodiment, elongate body 22a may have a maximum diameter of about 0.050 inches at a proximal portion 21a when used without an outer needle such as a Brockenbrough needle. In such an embodiment, portion 24a can be up to about 0.032 inches in diameter.

In an exemplary embodiment, after elongate body 22a is formed, radiopaque markers (25, FIG. 3) may be slidably coupled to portion 24a to form an imagable section (24, FIG. 3) of elongate body 22a. Imagable section (24, FIG. 3) has a substantially circular cross-section having a diameter of about 0.008 inches according to one embodiment. Thus, at least a portion of imagable section (24, FIG. 3) has a cross-sectional area smaller than the maximum cross-sectional area defined in portion 21a of elongate body 22a. In an exemplary embodiment, radiopaque markers (25, FIG. 3) coupled to portion 24a may be bands that have inner diameters greater than the diameter of portion 24a. Radiopaque marker bands, for example, may have inner diameters greater than about 0.008 inches and less than about 0.011 inches. Outer diameters of radiopaque marker bands may be greater than about 0.010 inches.

One or more radiopaque markers (25, FIG. 3) may be mounted to portion 24a of elongate body 22a by various coupling processes. Radiopaque markers, for example, may be mounted by adhesives, swaging, crimping, welding, or printing. Swaging techniques, for instance, include plastically deforming radiopaque markers (25, FIG. 3) using high pressure so that markers are crimped onto portion 24. Adhesive bonding methods may use low viscosity adhesives, such as cyanoacrylate, which is typically sold under trademarks like “Superglue” and “Krazy Glue.” In an exemplary embodiment, platinum/10% iridium radiopaque marker bands (25, FIG. 3) using materials available from Johnson Matthey in West Chester, Pa. are attached to imagable section (24, FIG. 3) with wicking grade cyanoacrylate adhesive from Henkel Loctite Corporation in Rocky Hill, Conn. In another embodiment, radiopaque markers is (25, FIG. 3) are slidably coupled to portion 24a without the use of adhesives or are otherwise applied.

FIGS. 4B and 4C illustrate enlarged views of tapered transitions from portion 21a of elongate body 22a to adjacent portion 24a and tapered distal section 28. Tapered transition from portion 21a to adjacent portion 24a may span a length of about 0.05 inches, for example, and taper from its maximum diameter to about 0.008 inches. As described above, portion 24a and pointed tip 29 are generally formed by centerless grinding such that portion 24a and pointed tip 29 have diameters less than the maximum diameter of elongate body 22a. Tapered distal section 28 has a portion with a diameter equal to or less than about 0.008 inches which tapers to a sharp pointed tip 29.

Referring now to FIGS. 4A and 5, additional aspects of a procedure for forming transseptal guidewire 20 are shown. In an exemplary embodiment, after radiopaque markers (25, FIG. 3) are coupled to imagable section 24 of elongate body 22a, at least a segment of portion 24a is ovalized or pressed using a mechanism such as a toggle press to form at least a portion of the end section 26. End section 26 is about ½ inch long and has a substantially non-circular cross-section distal of imagable section 24 and proximal of tapered distal section 28. As described in further detail below, when end section 26 is ovalized or otherwise pressed or formed, end section 26 has a first dimension (such as a width) in a direction transverse to longitudinal axis 1 that is larger than a second dimension (such as a thickness) in a second direction transverse to longitudinal axis 1. The first dimension of end section 26 is larger than the diameter of imagable section 24 to minimize the risk of radiopaque marker bands (25, FIG. 3) migrating from or falling off elongate body 22a.

The first dimension, for example, may have a width between about 0.008 and about 0.014 inches and greater than the inner and outer diameters of radiopaque marker bands (25, FIG. 3). Conversely, the inner diameters or dimension of radiopaque marker bands (25, FIG. 3) are preferably no larger than, and more preferably are smaller than, the first dimension of end section 26 such that the bands are restrained from passing over or along the end section 26. Also, the outer circumference of the bands preferably does not exceed the maximum circumference or perimeter of end section 26. When the outer circumference of the bands does not exceed the maximum circumference or perimeter of end section 26, then the bands are apt to pass more easily through an aperture in the septum formed by the end section 26. This reduces the interference as the trannseptal guidewire is advanced.

Due to ovalization or pressing or other forming, the second dimension of end section 26 is smaller than the diameter of imagable section 24 and may have a thickness, for example, less than about 0.008 inches such as about 0.005 inches. Accordingly, end section 26 of the transseptal guidewire 20 is thinner and therefore more flexible than proximal portion 21a, imagable section 24, and tapered distal section 28 in a direction of curvature about an axis parallel to the first dimension. In other words, the end section 26, like an “I-beam,” is more flexible in one direction (about an axis parallel to the first dimension) as compared to another direction (about an axis parallel to the second dimension).

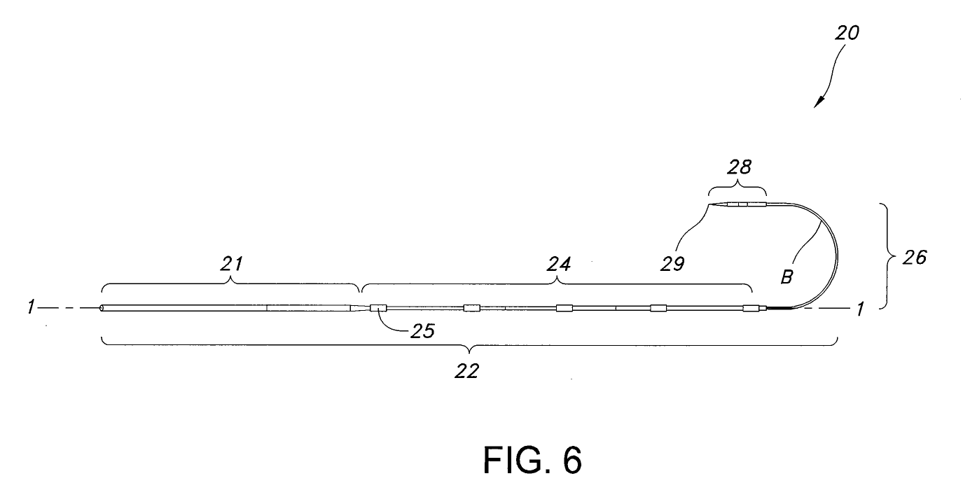

Referring now to FIGS. 5 and 6, end section 26 of transseptal guidewire 20 is biased to a curved configuration by a heat curving process or other forming process. For example, end section 26 may be treated at an elevated temperature, such as about 500 degrees Centigrade, for a set duration, such as about 10 seconds, to curve an otherwise linear superelastic nitinol wire. At least a portion of end section 26 is curved by a fixture and then cooled to retain the flexibility of the curved configuration. Thus, when end section 26 of transseptal guidewire 20 is not constrained within the lumen (13, FIG. 2) of outer needle (14, FIG. 2), end section 26 has an essentially non-traumatic conformation, such as a helical, curved, or hook shape.

For example, the radius “B” of the loop that forms the curved configuration can be about 0.125 inches or the diameter may be about 5-8 mm, though other dimensions are optionally selected. When the tapered distal section 28 is enclosed within the lumen (13, FIG. 2) of outer needle (14, FIG. 2), the entire length of the transseptal guidewire 20 is substantially straight and parallels longitudinal axis 1 of outer needle (14, FIG. 2).

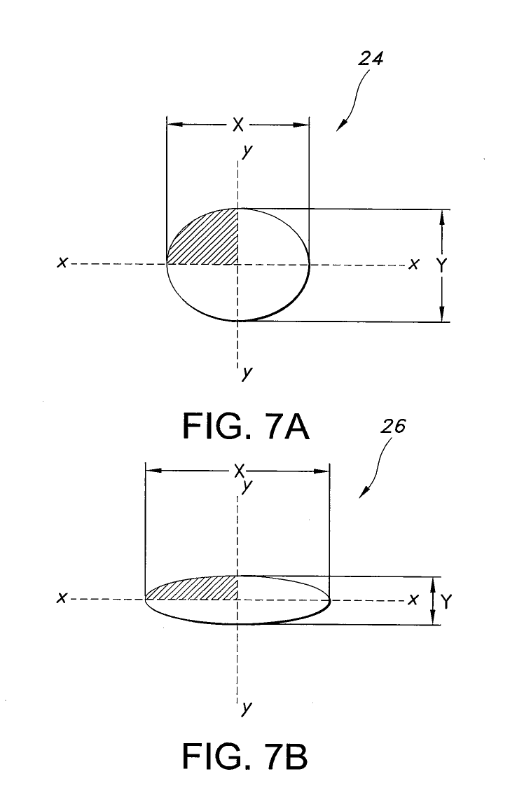

FIGS. 7A and 7B illustrate cross-sectional views of transseptal guidewire 20 taken along lines A-A of imagable section 24 and lines B-B of end section 26 shown in FIG. 5. As shown in FIG. 7A, imagable section 24 has a substantially circular cross-section having a cross-sectional area less than the maximum cross-sectional area of elongate body (22, FIG. 6). In an exemplary embodiment, the diameter of imagable section 24 is less than the maximum diameter of elongate body (22, FIG. 6), preferably a diameter of about 0.008 in. When radiopaque marker bands (25, FIG. 6) are coupled to the imagable section 24, inner diameters of radiopaque marker bands (25, FIG. 6) are positioned adjacent the circumference or perimeter of imagable section 24.

As shown in FIGS. 5 and 7B, end section 26 is ovalized or pressed or otherwise formed from a portion (24a, FIG. 4a) of elongate body (22a, FIG. 4a) such that end section 26 has a substantially non-circular cross-section. End section 26 has a first dimension (such as a width) that is greater than the diameter of imagable section 24. As described above, and according to one exemplary embodiment, the first dimension X is between about 0.008 inch and about 0.014 inch, and is preferably about 0.011 inch. The second dimension Y of end section 26 is smaller than the diameter of imagable section 24 and may have a thickness less than about 0.008 inch, for example, about 0.005 inch. In an exemplary embodiment, radiopaque marker band (25, FIG. 6) has a circumference not exceeding the maximum circumference or perimeter of end section 26 such that radiopaque marker band (25, FIG. 6) can pass without substantial resistance through an aperture formed by the end section 26. Also, an inner dimension such as an inner diameter of the band is preferably smaller than the largest dimension of the end section 26 so that the band may be retained on imagable section 24 and not fall off the transseptal guidewire 20 by passing through or along end section 26. Radiopaque marker bands (25, FIG. 6) may have a pull force greater or equal to about 3 Newtons in compliance with ISO 11070, thereby securing the bands to imagable section 24.

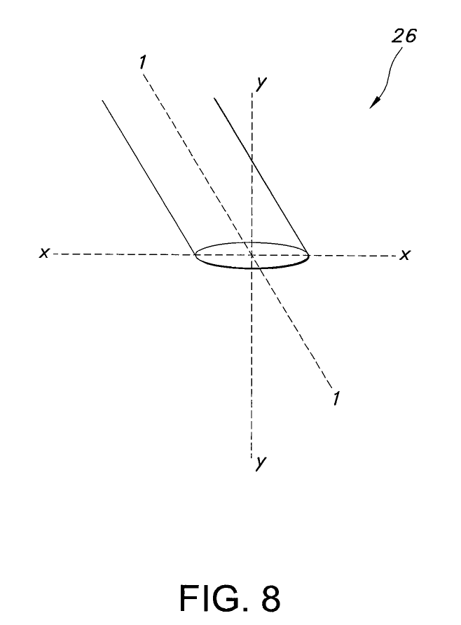

Referring now to FIGS. 7B and 8, when end section 26 is ovalized according to the illustrated embodiment, first dimension X of end section 26 is formed in a first direction transverse to longitudinal axis 1 of elongate body (22, FIG. 5) and second dimension Y is formed in a second direction transverse to longitudinal axis 1. First dimension X is larger than second dimension Y, thus end section 26 is thinner in thickness and more flexible in at least one direction as compared to proximal portion 21, imagable section 24, and tapered distal portion 28 of transseptal guidewire 20.

The exemplary embodiments of end section 26 are illustrated schematically as having a portion with a cross-sectional shape that is like an oval. This oval shape may be formed by pressing or other techniques. It is contemplated that this shape may be something other than an oval as well, while still maintaining first and second respective dimensions. For example, the shape may be flattened or somewhat rectangular. It may also take any other geometric shape. In any shape selected, however, the subject portion of end section 26 preferably serves at least one of the functions of retaining radiopaque bands, promoting increased flexibility in at least one direction, and providing an outer perimeter close to the outer perimeter of the radiopaque bands.

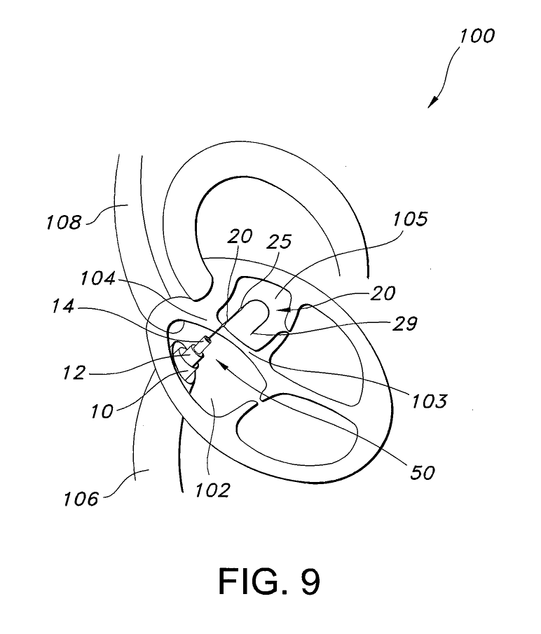

Referring now to FIGS. 1, 2, 6, and 9, methods of perforating an intra-atrial septum 104 and confirming traversal of the intra-atrial septum 104 to treat, for example, patent foramen ovale (PFO) or to gain access to the left atrium 105 are illustrated. As shown in FIGS. 1 and 9, one exemplary method includes the step of introducing an intravascular sheath 10 in a vessel such as the inferior vena cava 106 or superior vena cava 108 to access the chamber of right atrium 102. In an embodiment, the distal end of sheath 10 is tapered to enhance advancement of the sheath 10 though the intra-atrial septum 104 after perforating intra-atrial septum 104.

Referring to FIG. 1, after the sheath 10 is properly positioned in the right atrium 102, dilator 12 and outer needle 14.of transseptal trocar device 50 are advanced distally toward the intra-atrial septum 104. The distal end of outer needle 14 is positioned against fossa ovalis 103 at the perforate site and pushed against fossa ovalis 103 until some tenting of the fossa ovalis 103 is caused. The tenting should be sufficient to correctly identify, preferably by visualization, the perforate site in the intra-atrial septum 104. Alternatively, visualization techniques such as intracardiac echocardiography (ICE) or magnetic resonance imaging (MRI) can be used that may work without tenting.

Once outer needle 14 is positioned, transseptal guidewire 20 is advanced relative to the outer needle 14 through the septum 104. The perforation force of transseptal guidewire 20 is less than or equal to the perforation force of currently available transseptal needles such as a Brockenbrough needle. According to one embodiment, at its most distal position, about 10 mm of the transseptal guidewire 20 should extend from the distal end of outer needle 14. Alternatively, the most distal position could be extended about 30 mm to 50 mm, e.g., 3-5 cm, if end section 26 of transseptal guidewire 20 has a hook shape, as is shown in FIG. 6. Thus, after perforation of fossa ovalis 103, a portion of transseptal guidewire 20 may be extended into left atrium 105 to confirm that it is in the left atrium 105. In another embodiment, curved portion of transseptal guidewire 20 may be advanced such that the curved portion is adjacent the entrance to one of the pulmonary veins (not shown) in the left atrium 105.

In an embodiment of this procedure, as elongate body 22 is advanced through outer needle 14, the straight configuration of transseptal guidewire 20 shown in FIG. 2, transitions to curved configuration of end section 26. A portion of end section 26 extends into the left atrium 105 such that pointed tip 29 of the tapered distal section 28 curves back toward the intra-atrial septum 104 as shown in FIG. 9. Imagable section 24 proximal of end section 26 may then be advanced into the left atrium 105 and imaged by means of at least one radiopaque marker 25 coupled to the imagable section 24. Radiopaque markers 25 may be restricted from movement along longitudinal axis 1 of the transseptal guidewire 20 and exclusively positioned along imagable section 24 by ovalizing or otherwise pressing or forming end section 26 as shown in FIG. 7B or otherwise changing the cross-sectional shape of the end section 26. When radiopaque markers 25 are positioned within the left atrium 105, traversal of the intra-atrial septum 104 and the location of transseptal guidewire 20 are confirmed by imaging of radiopaque markers 25.

In an embodiment of this procedure, outer needle 14 follows the path of transseptal guidewire 20 through the septum 104. Alternatively, because of the added stiffness provided by outer needle 14, transseptal guidewire 20, dilator 12, and sheath 10 can be advanced through septum 104. The motion of the transseptal guidewire 20 may be forward, vibrating, reciprocating, linear, or rotational, for example. In one embodiment, movement of the transseptal guidewire 20 is accomplished manually, thus providing easier manipulation for the surgeon.

As shown in FIGS. 1 and 9, once the pointed tip 29 of the transseptal guidewire 20 is positioned within the septum 104, fossa ovalis 103 tissue provides support to the transseptal guidewire 20 until sheath 10, dilator 12, and/or outer needle 14 is delivered into the left atrium 105. According to standard catheterization procedures, once sheath 10 and/or dilator 12 is positioned in the left atrium 105, other components of the transseptal trocar device 50, for example, the transseptal guidewire 20, outer needle 14, and dilator 12 can be retracted and the sheath 10 can be used to deliver implants, for example, such as an atrial occluder for the treatment of a patent foramen ovale, sutures, or other intracardiac therapeutic devices. In an embodiment of this procedure, the transseptal guidewire 20 is left in the left atrium 105 to maintain the perforate site as well as to image the operative area by radiopaque markers 25, or to act as a guidewire for delivery of over-the-wire devices into the left atrium. In another embodiment, the transseptal guidewire 20 is withdrawn, e.g., into the outer needle 14.

The method for transseptal perforation using the transseptal device described herein offers several significant advantages. For example, when using the devices and methods according to exemplary embodiments of the invention, inadvertent contact of the transseptal guidewire 20 with the left atrial free wall immediately after the septum 104 is perforated does not result in damage to or perforation of the left atrial free wall because the end section 26 of the transseptal guidewire 20 is flexible and/or biased to a curved configuration when fully extended from the distal end of outer needle 14. In other words, the flexibility and/or curvature of the end section renders it atraumatic.

When the end section 26 of the transseptal guidewire 20 contacts the left atrial free wall or pulmonary vein, for example, end section 26 of transseptal guidewire 20 harmlessly bends rather than perforates the left atrial free wall. In one embodiment, the end section 26 of the transseptal guidewire 20 bends because of the enhanced flexibility of the ovalized end section 26, as described above. In an embodiment, perforation of the left atrial wall is avoided by modifying the shape of the end section 26 of transseptal guidewire 20 to form, for example, a hook or a bend. In yet another embodiment, end section 26 of transseptal guidewire 20 may be advanced into one of the pulmonary veins in the left atrium 105 and straightened by advancing a transseptal introducer, such as dilator 12 or sheath 10, over end section 26.

Another advantage of the transseptal trocar device embodiments described herein is the ability of the device to perforate through thick septum such as septum secundum. The transseptal trocar devices according to the invention can also be used for remote suturing of a patent foramen ovale or other defects that may be accessed vascularly. This is possible, for example, because the fit between the outer needle 14 and the guidewire 20, especially when provided with an ovalized end section, promotes the column strength of the guidewire and reduces the bending or buckling tendency of the guidewire. This fit, promoted by the ovalized end section, improves the ability of the guidewire to perforate tougher tissue yet, when extended from the end of the needle 14, becomes relatively atraumatic.

In an exemplary embodiment, the pointed tip of the guidewire 20 is significantly sharper and/or smaller than the tip of the transseptal outer needle 14. Thus, the guidewire 20 is able to perforate through the fossa ovalis 103 with less force. When needle 14 punctures the fossa ovalis 103, the needle 14 continues on a path towards the lateral wall of the left atrium. According to exemplary embodiments described herein, however, when the guidewire 20 is extended from the tip of the transseptal outer needle 14, guidewire 20 prevents the needle 14 from puncturing the lateral wall of the left atrium.

By way of example, the flexible members are manufactured using nickel-titanium material, such as superelastic nitional, or other shape memory alloy materials. The nickel-titanium wire, when properly manufactured, exhibits elastic properties for the wire to be manipulated (e.g., bent) by an operator and then returned to substantially the same shape the wire possessed prior to it being manipulated. Thus, transseptal guidewire 20 does not kink or buckle during use with transseptal trocar device 50.

In an exemplary embodiment, components of transseptal trocar device 50 are passed through a straightener and optional hemostatic Y adapter (not shown) without resistance. The hemostatic Y adapter may be used to supply contrast imaging fluid through the sheath 10, dilator, and/or needle 14. Alternatively, the Y adapter may be coupled to a pressure monitor to measure atrial pressure change when the intra-atrial septum 104 is perforated.

In yet another embodiment, transseptal trocar device 50 may be provided in a sterilized kit which includes intravascular sheath 10, dilator 12, outer needle 14, transseptal guidewire 20, and the hemostatic Y valve. The components of the kit may be packaged in a tyvek/polymylar pouch for one time use such that the transseptal trocar device 50 may be disposable after a surgical procedure. Additional aspects of the Y adapter and transseptal catheterization methods are described in U.S. Pat. No. 5,312,341, U.S. Patent Publication 2006/0064062, and U.S. Patent Publication 2005/0101984, which are incorporated herein fully by reference.

Accordingly, a surgical device is provided, according to exemplary embodiments of the invention, that reduces the risk of inadvertent perforation or trauma in transseptal procedures with the added benefit of confirming the puncture location prior to dilation. In particular, such embodiments provide accurate placement and safe access to the left atrium through the atrial septum. The device, according to exemplary embodiments, preferably performs with commercially available transseptal needle systems and allows for safer and easier penetration of a transseptal needle through the atrial septum.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A transseptal guidewire configured to perforate the intra-atrial septum having an elongated body, an end section, and a tapered distal section, with at least a portion of the end section having a first dimension in a first direction transverse to the longitudinal axis of the elongated body that is larger than a second dimension in a second direction transverse to the longitudinal axis.

2. The transseptal guidewire of claim 1, the elongated body having a portion, proximal of the end section, with a substantially circular cross section.

3. The transseptal guidewire of claim 1, where the end section is biased in a curved configuration.

4. The transseptal guidewire of claim 1, a maximum diameter of the elongated body being about 0.050 or about 0.015 in if used in conjunction with an outer needle.

5. The transseptal guidewire of claim 1, the tapered distal section terminating at a pointed tip.

6. The transseptal guidewire of claim 5, the pointed tip having a substantially circular cross-section.

7. The transseptal guidewire of claim 1, at least a portion of the end section being ovalized.

8. The transseptal guidewire of claim 7, the ovalized portion of the end section being about ½ in long.

9. The transseptal guidewire of claim 1, the first dimension being between about 0.008 and about 0.014 in.

10. The transseptal guidewire of claim 9, the first dimension being about 0.011 in.

11. The transseptal guidewire of claim 1, the second dimension being less than about 0.008 in.

12. The transseptal guidewire of claim 11, the second dimension being about 0.005 in.

13. The transseptal guidewire of claim 1 further comprising an imagable section proximal of the end section.

14. The transseptal guidewire of claim 13, at least a portion of the imagable section having a cross-sectional area smaller than a maximum cross-sectional area of the elongated body.

15. The transseptal guidewire of claim 14, the imagable section having a substantially circular cross-section.

16. The transseptal guidewire of claim 15, the imagable section having a diameter of about 0.008 in.

17. The transseptal guidewire of claim 13 further comprising a tapered transition from the imagable section to an adjacent portion of the elongated body.

18. The transseptal guidewire of claim 13, the imagable section comprising at least one radiopaque marker.

19. The transseptal guidewire of claim 18, the imagable section comprising between three and five radiopaque markers.

20. The transseptal guidewire of claim 18, the at least one radiopaque marker comprising a band.

21. The transseptal guidewire of claim 20, the band being mounted by adhesive.

22. The transseptal guidewire of claim 21, wherein the adhesive comprises a low viscosity cyanoacrylate.

23. The transseptal guidewire of claim 20, the band being mounted by swaging.

24. The transseptal guidewire of claim 20, the band having a circumference not exceeding the maximum circumference or perimeter of the end section.

25. The transseptal guidewire of claim 24, the band having an outer diameter greater than about 0.010 in.

26. The transseptal guidewire of claim 20, the band having an inner diameter smaller than the first dimension of the end section.

27. The transseptal guidewire of claim 26, the band having an inner diameter less than about 0.011 in.

28. The transseptal guidewire of claim 18, the radiopaque marker comprising a platinum/iridium alloy.

29. The transseptal guidewire of claim 1, wherein the elongate body comprises superelastic nitinol material.

30. A transseptal guidewire configured to perforate the intra-atrial septum having an elongated body with an end section biased to a curved configuration and a tapered distal section, the elongate body comprising an imagable section proximal of the end section, the imagable section comprising at least one radiopaque marker, and at least a portion of the end section having a first dimension in a first direction transverse to a longitudinal axis of the elongated body that is larger than a second dimension in a second direction transverse to the longitudinal axis.

31. The transseptal guidewire of claim 30, wherein the at least one radiopaque marker is exclusively positioned along the imagable section.

32. A method of fabricating a transseptal guidewire comprising the steps of:
coupling at least one radiopaque marker to an elongate body to form an imagable section of the elongate body;
ovalizing at least a portion of an end section of the elongate body distal of the imagable section; and
heat curving at least a portion of the end section to a provide a curved configuration.

33. The method of claim 32, further comprising the step of centerless grinding the elongate body.

34. The method of claim 33, wherein said centerless grinding step comprises grinding the elongate body at the imagable section to a diameter less than a maximum diameter of the elongated body.

35. The method of claim 33, wherein said centerless grinding step comprises forming a pointed tip at a distal end of the tapered distal section.

36. The method of claim 32, wherein said coupling step comprises adhering the at least one radiopaque marker with a low viscosity adhesive.

37. The method of claim 32, wherein said ovalizing step comprises forming a first dimension in a first direction transverse to a longitudinal axis of the elongated body and a second dimension in a second direction transverse to the longitudinal axis, the first dimension being larger than the second dimension.

38. The method of claim 32, wherein said ovalizing step comprises pressing the portion of the end section.

39. A method of confirming traversal of an intra-atrial septum comprising the steps of:
perforating the intra-atrial septum from a right atrium of a heart to a left atrium of the heart using a transseptal guidewire;
extending at least a portion of an end section of the transseptal guidewire into the left atrium; and
imaging an imagable section of the transseptal guidewire proximal of the end section in the left atrium by means of at least one radiopaque marker coupled to the transseptal guidewire at the imagable section.

40. The method of claim 39, further comprising the step of restricting movement of the at least one radiopaque marker along a longitudinal axis of the transseptal guidewire with an ovalized portion of the end section of the transseptal guidewire.

41. The method of claim 39, said imaging step comprising imaging a plurality of radiopaque markers in the left atrium.

42. A method of providing vascular access to the left atrium comprising the steps of:
perforating the intra-atrial septum from a right atrium of a heart to a left atrium of the heart using a transseptal guidewire;
extending at least a portion of an end section of the transseptal guidewire into the left atrium;
imaging an imagable section of the transseptal guidewire proximal of the end section in the left atrium by means of at least one radiopaque marker coupled to the transseptal guidewire at the imagable section;
advancing the transseptal guidewire into one of the pulmonary veins to confirm location; and
advancing a transseptal introducer over the transseptal guidewire into the left atrium.

43. The method of claim 39 or 42, wherein said extending step is performed such that a pointed tip of the distal section curves toward the intra-atrial septum.
